(19) United States
(12) Patent Application Publication
(10) Pub. No.: US 2025/0327618 A1
(43) Pub. Date: Oct. 23, 2025

(54) ETHANE RECOVERY SYSTEM SUITABLE FOR RICH GAS WITH HIGH CARBON DIOXIDE CONTENT AND RECOVERY METHOD THEREFOR

(71) Applicant: Jiangsu Urban and Rural Construction Vocational College, Changzhou (CN)

(72) Inventors: Le Li, Changzhou (CN); Shaoyu Zhang, Changzhou (CN); Meijun Han, Changzhou (CN); Zhifan Chen, Changzhou (CN); Linyan Zhang, Changzhou (CN); Xinyan Zhou, Changzhou (CN)

(21) Appl. No.: 19/182,432

(22) Filed: Apr. 17, 2025

(30) Foreign Application Priority Data
Apr. 19, 2024 (CN) 202410475637.X

(51) Int. Cl. F25J 3/02

(52) U.S. Cl. CPC F25J 3/0238; F25J 3/0233; F25J 3/0295; F25J 2200/06; F25J 2205/50; F25J 2215/60; F25J 2215/62; F25J 2230/60; F25J 2235/60; F25J 2270/08; F25J 2270/12; F25J 2270/60

(57) ABSTRACT

The disclosure relates to the technical field of ethane recovery systems, and in particular to an ethane recovery system suitable for a rich gas with high carbon dioxide content and a recovery method therefor. The recovery system includes a first pre-cooling cold box, a second pre-cooling cold box, a subcooling cold box, a low temperature separator, an absorption tower, a tower top separator and a demethanizer. According to the disclosure, freezing and blockage problems occurring in conventional demethanizers when CO₂ content ≥2 mol % are effectively solved. Meanwhile, an operation pressure of the demethanizer is ≥300 KPa compared with that of the absorption tower, significantly reducing power consumption of an output compressor, and making an ethane recovery device more energy efficient, and the disclosure is suitable for an ethane recovery device for a medium and high pressure rich gas with high carbon dioxide content.

### CROSS-REFERENCE TO RELATED APPLICATION

This application claims priority of Chinese Patent Application No. 202410475637.X, filed on Apr. 19, 2024, the entire contents of which are incorporated herein by reference.

### TECHNICAL FIELD

The disclosure relates to the technical field of ethane recovery systems, and in particular to an ethane recovery system suitable for a rich gas with high carbon dioxide content and a recovery method therefor.

### BACKGROUND

With the constant advancement of quality and efficiency improvement projects in major oilfields, oil and gas fields with high carbon dioxide content are increasingly developed, and the economic potential from natural gas ethane and heavy hydrocarbon products is continuously explored. Therefore, the development and research of ethane recovery processes for a rich gas with high carbon dioxide content are extremely important.

As shown in FIG. 2, ethane recovery procedures in some of the existing typical dry gas recycle processes are characterized in that a part of the external dry gas is subcooled into a liquid phase, which is used as a feed material of the top of a demethanizer, and the ethane recovery rate is increased by means of enhancing the reflux and liquefaction rate of the external dry gas. When a higher CO₂ content is present in the feed gas, the CO₂ freezing and blockage problems are solved by either increasing the liquid phase volume from a low temperature separator mixed into the second stream of feed material of the demethanizer or by pressurizing the demethanizer. However, this results in a large amount of propane and heavy hydrocarbons being mixed into the external dry gas, causing losses of the heavy hydrocarbons. Additionally, the pressurization of the demethanizer reduces expansion refrigeration capacity, increases external cooling demands, and substantially increases compression power of external refrigeration devices, thus leading to a significant increase in energy consumption of system, and a reduced economic benefit in the ethane recovery device.

### SUMMARY

The technical problem to be solved by the disclosure is that: to solve the problems of some of the existing dry gas recycle processes in which the CO₂ content of the feed gas is ≥2 mol %, the total compression power consumption of the device is large, there are no available pressure differentials in a case that the pressure of the feed gas is almost equal to the output pressure, and the large-scale medium and high pressure rich gas requires to be treated, an ethane recovery system suitable for a rich gas with high carbon dioxide content and a recovery method therefor are provided.

The technical solution employed in the disclosure to solve the technical problem is: an ethane recovery system suitable for a rich gas with high carbon dioxide content, which includes a first pre-cooling cold box, a second pre-cooling cold box, a subcooling cold box, a low temperature separator, an absorption tower, a tower top separator and a demethanizer.

A pre-cooling input end of the first pre-cooling cold box and a pre-cooling input end of the second pre-cooling cold box are in communication with an external feed gas, and a pre-cooling output end of the first pre-cooling cold box is in communication with a pre-cooling input end of the subcooling cold box; a pre-cooling output end of the subcooling cold box is in communication with a top of the absorption tower, and a pre-cooling output end of the second pre-cooling cold box is in communication with the low temperature separator; and a gas phase end of the low temperature separator is in communication with a middle of the absorption tower via a first turbo expander expansion end, and a liquid phase end of the low temperature separator is separately in communication with a bottom of the absorption tower and the pre-cooling output end of the first pre-cooling cold box.

A gas phase end at the top of the absorption tower is in communication with a heat exchange input end of the subcooling cold box, and a heat exchange output end of the subcooling cold box is separately in communication with a heat exchange input end of the first pre-cooling cold box and a heat exchange input end of the second pre-cooling cold box; a heat exchange output end of the first pre-cooling cold box and a heat exchange output end of the second pre-cooling cold box are in communication with an input end of an output compressor via a second turbo expander pressurized end, and an output end of the output compressor is in communication with an output end of an air cooler; the output end of the air cooler is separately in communication with an outside and the pre-cooling input end of the first pre-cooling cold box, and the pre-cooling output end of the first pre-cooling cold box is in communication with the pre-cooling input end of the second pre-cooling cold box; the pre-cooling output end of the second pre-cooling cold box is in communication with the top of the absorption tower, and the bottom of the absorption tower is in communication with an upper part of the demethanizer via a first liquid phase pump; and a gas phase end at a top of the demethanizer is in communication with the heat exchange input end of the subcooling cold box, and the heat exchange output end of the subcooling cold box is in communication with the tower top separator.

A gas phase end of the tower top separator is in communication with the heat exchange input end of the subcooling cold box, and the heat exchange output end of the subcooling cold box is in communication with the heat exchange input end of the first pre-cooling cold box; the heat exchange input end of the first pre-cooling cold box is in communication with the input end of the output compressor, and a liquid phase end of the tower top separator is in communication with the upper part of the demethanizer via a second liquid phase pump; and a condensate product discharge end is arranged at a bottom of the demethanizer. Compared with the prior art, in the solution, a low-temperature rectification section of the demethanizer is replaced with the absorption tower and the tower top separator of the demethanizer, so that CO₂ content in the gas phase entering a low temperature zone is reduced, and most of CO₂ is diverted to a stripping section of the demethanizer, effectively solving freezing and blockage problems occurring in conventional demethanizers when CO₂ content ≥2 mol %. Meanwhile, the operation pressure of the demethanizer is ≥300 KPa compared with that of the absorption tower, significantly reducing power consumption of the output compressor, and making the ethane recovery device more energy efficient. The disclosure is suitable for an ethane recovery device for a medium and high pressure rich gas with high carbon dioxide content.

In some preferred embodiments, the first liquid phase pump is in communication with the second pre-cooling cold box via an external pipeline and is in communication with the bottom of the demethanizer.

In some preferred embodiments, the bottom of the demethanizer is in communication with the second pre-cooling cold box via an external pipeline and is in communication with the bottom of the demethanizer.

A recovery method using the ethane recovery system suitable for a rich gas with high carbon dioxide content includes the following specific operation steps. A feed gas is divided into two paths. A first path of feed gas passes through the first pre-cooling cold box for pre-cooling, and then mixes with a part of a liquid in the low temperature separator. A mixed gas enters the subcooling cold box for subcooling, and then is throttled and cooled, followed by entering the upper part of the absorption tower.

A second path of feed gas passes through the second pre-cooling cold box for pre-cooling before entering the low temperature separator. A gas phase separated by the low temperature separator enters the first turbo expander expansion end for pressure and temperature reduction before entering the middle of the absorption tower, and a part of a liquid phase separated by the low temperature separator enters the bottom of the absorption tower.

A part of external dry gas before being throttled and cooled passes through the first pre-cooling cold box and the subcooling cold box for heat exchange and cooling, followed by entering the top of the absorption tower.

The liquid phase at the bottom of the absorption tower is divided into two paths after being pressurized by the first liquid phase pump, with a first path of the liquid phase being throttled and cooled before entering the upper part of the demethanizer, and a second path of the liquid phase being throttled and cooled before entering the second pre-cooling cold box for heat exchange and warming, followed by entering the middle of the demethanizer.

The gas phase at the top of the demethanizer passes through the subcooling cold box for heat exchange and warming, followed by entering the tower top separator of the demethanizer. The gas phase separated by the tower top separator of the demethanizer sequentially passes through the subcooling cold box and the first pre-cooling cold box for heat exchange and warming, and then passes through the output compressor for pressurization, and finally passes through the air cooler for cooling and then is outputted. The liquid phase separated by the tower top separator of the demethanizer enters the top of the demethanizer after pressurization by the second liquid phase pump.

The gas phase at the top of the absorption tower is divided into two paths after heat exchange and warming by the subcooling cold box, which separately enter the first pre-cooling cold box and the second pre-cooling cold box for heat exchange and warming, followed by merging. A merged gas phase is pressurized in the second turbo expander pressurized end and merges with the gas phase at the top of the demethanizer, followed by entering the output compressor for pressurization. A condensate product at the bottom of the demethanizer enters subsequent fractionation processing units including a dethanizer for processing.

The beneficial effects of the disclosure are as follows. According to the ethane recovery system suitable for a rich gas with high carbon dioxide content and the recovery method therefor, when in use, a low-temperature rectification section of the demethanizer is replaced with the absorption tower and the tower top separator of the demethanizer, so that CO₂ content in the gas phase entering a low temperature zone is reduced, and most of CO₂ is diverted to a stripping section of the demethanizer, effectively solving freezing and blockage problems in conventional demethanizers when CO₂ content ≥2 mol %. Meanwhile, the operation pressure of the demethanizer is ≥300 KPa compared with that of the absorption tower, significantly reducing power consumption of the output compressor, and making the ethane recovery device more energy efficient, and the disclosure is suitable for an ethane recovery device for a medium and high pressure rich gas with high carbon dioxide content. In the disclosure, an ethane recovery method for a medium and high pressure rich gas with high carbon dioxide content is developed, achieving an ethane recovery rate up to 95% under high CO₂ content conditions. The total compression power consumption of the device is reduced, economic and social benefits of the ethane recovery device are improved, and the problems of some existing dry gas recycle processes, in which the CO₂ content of feed gas ≥2 mol %, the total compression power consumption of the device is large, there are no available pressure differentials, and the large-scale medium and high pressure rich gas requires to be treated, are avoided.

### BRIEF DESCRIPTION OF THE DRAWINGS

The disclosure is further described by reference to the accompanying drawings and examples below.

FIG. 1 is a schematic structural diagram of the disclosure; and

FIG. 2 is a schematic structural diagram of the prior art.

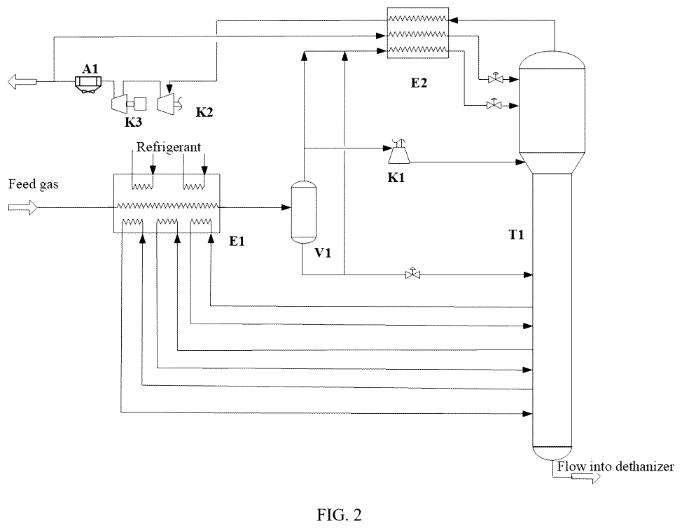

Reference numerals and denotations thereof: E1—first pre-cooling cold box, V1—low temperature separator, E2—second pre-cooling cold box, V2—tower top separator, K1—first turbo expander expansion end, E3—subcooling cold box, T1—adsorption tower, T2—demethanizer, A1—air cooler, K2—second turbo expander pressurized end, K3—output compressor, P1—first liquid phase pump, and P2—second liquid phase pump.

### DETAILED DESCRIPTION

The disclosure is further described by reference to the examples below.

The disclosure is not limited to the following specific embodiments. Those of ordinary skill in the art may implement the disclosure through various other embodiments according to the disclosed content of the disclosure. Any simple modifications or alterations made on the basis of the design structure and concepts of the disclosure shall fall within the scope of protection of the disclosure. It is to be noted that the following examples and features in the examples may be combined with each other without conflict.

In the description of the disclosure, it is to be understood that, the orientation or state relations indicated by the terms “center”, “longitudinal”, “crosswise”, “up”, “down”, “front”, “rear”, “left”, “right”, “vertical”, “horizontal”, “top”, “bottom”, “inner”, “outer”, etc. are based on those shown in the accompanying drawings and merely for the ease of describing the disclosure and simplifying the description, rather than indicating or implying that the device or element referred to must be in a specific orientation or constructed and operated in a specific orientation, and therefore cannot be interpreted as limiting the disclosure. Moreover, the terms “first” and “second” are only used to describe the objective, not to be understood as indicating or implying relative importance or indicating the quantity of technical features indicated. Therefore, a feature defined with “first” and “second” may include one or more of these features explicitly or implicitly. In the description of the disclosure, unless otherwise stated, “a plurality of” means two or more.

In the description of the disclosure, it is to be noted that unless otherwise clearly specified and limited, the terms “mounted”, “connection”, and “be connected to” are to be understood in a broad sense. For example, the connection can be fixed connection, detachable connection, integral connection, mechanical connection, electrical connection, direct connection, indirect connection through an intermediate medium, or connection between two components. For those of ordinary skill in the art, the specific meanings of the above terms in the disclosure can be understood according to specific circumstances.

### Example 1

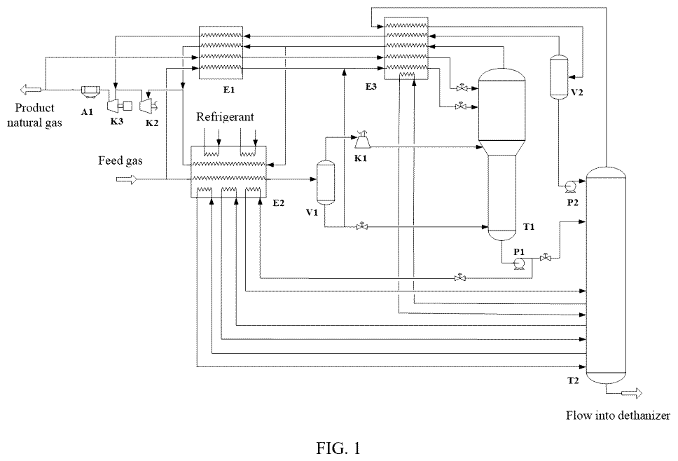

As shown in FIG. 1, an ethane recovery system suitable for a rich gas with high carbon dioxide content includes a first pre-cooling cold box E1, a second pre-cooling cold box E2, a subcooling cold box E3, a low temperature separator V1, an absorption tower T1, a tower top separator V2 and a demethanizer T2.

A pre-cooling input end of the first pre-cooling cold box E1 and a pre-cooling input end of the second pre-cooling cold box E2 are in communication with an external feed gas, and a pre-cooling output end of the first pre-cooling cold box E1 is in communication with a pre-cooling input end of the subcooling cold box E3; a pre-cooling output end of the subcooling cold box E3 is in communication with a top of the absorption tower T1, and a pre-cooling output end of the second pre-cooling cold box E2 is in communication with the low temperature separator V1; and a gas phase end of the low temperature separator V1 is in communication with a middle of the absorption tower T1 via a first turbo expander expansion end K1, and a liquid phase end of the low temperature separator V1 is separately in communication with a bottom of the absorption tower T1 and the pre-cooling output end of the first pre-cooling cold box E1.

A gas phase end at the top of the absorption tower T1 is in communication with a heat exchange input end of the subcooling cold box E3, and a heat exchange output end of the subcooling cold box E3 is separately in communication with a heat exchange input end of the first pre-cooling cold box E1 and a heat exchange input end of the second pre-cooling cold box E2; a heat exchange output end of the first pre-cooling cold box E1 and a heat exchange output end of the second pre-cooling cold box E2 are in communication with an input end of an output compressor K3 via a second turbo expander pressurized end K2, and an output end of the output compressor K3 is in communication with an output end of an air cooler A1; the output end of the air cooler A1 is separately in communication with an outside and the pre-cooling input end of the first pre-cooling cold box E1, and the pre-cooling output end of the first pre-cooling cold box E1 is in communication with the pre-cooling input end of the second pre-cooling cold box E2; the pre-cooling output end of the second pre-cooling cold box E2 is in communication with the top of the absorption tower T1, and the bottom of the absorption tower T1 is in communication with an upper part of the demethanizer T2 via a first liquid phase pump P1; and a gas phase end at a top of the demethanizer T2 is in communication with the heat exchange input end of the subcooling cold box E3, and the heat exchange output end of the subcooling cold box E3 is in communication with the tower top separator V2.

A gas phase end of the tower top separator V2 is in communication with the heat exchange input end of the subcooling cold box E3, and the heat exchange output end of the subcooling cold box E3 is in communication with the heat exchange input end of the first pre-cooling cold box E1; the heat exchange output end of the first pre-cooling cold box E1 is in communication with the input end of the output compressor K3, and a liquid phase end of the tower top separator V2 is in communication with the upper part of the demethanizer T2 via a second liquid phase pump P2; and a condensate product discharge end is arranged at a bottom of the demethanizer T2.

The first liquid phase pump P1 is in communication with the second pre-cooling cold box E2 via an external pipeline and is in communication with the bottom of the demethanizer T2.

The bottom of the demethanizer T2 is in communication with the second pre-cooling cold box E2 via an external pipeline and is in communication with the bottom of the demethanizer T2.

An external gas is divided into two paths: the first path of gas phase accounting for 10%-15% of the total gas phase after being cooled and throttled in the first pre-cooling cold box E1 and the subcooling cold box E3 enters; and the second path of gas phase is outputted as a qualified natural gas product.

A feed gas is divided into two paths: the first path of gas phase accounting for 20%-25% of the total gas phase after being cooled and throttled in the first pre-cooling cold box E1 and the subcooling cold box E3 enters the upper part of the absorption tower T1; and the second path of gas phase after being pre-cooled in the second pre-cooling cold box E2 enters the low temperature separator V1.

A liquid phase at the bottom of the low temperature separator V1 is divided into two paths: the first path of the liquid phase accounting for 75%-95% of the total liquid phase is mixed with the first path of feed gas, and the mixed phase after being cooled and throttled in the subcooling cold box E3 enters the upper part of the absorption tower T1; and the second path of the liquid phase after being throttled and cooled enters the bottom of the absorption tower T1.

The liquid phase at the bottom of the absorption tower T1 is divided into two paths: the first path of the liquid phase accounting for 40%-60% of the total liquid phase after being throttled and cooled enters the upper part of the demethanizer T2; the second path of the liquid phase after being throttled and cooled enters the upper part of the absorption tower T1; and the second path of the liquid phase after being throttled and cooled enters the first pre-cooling cold box E1, and after being subjected to heat exchange and warming, enters the lower part of the absorption tower T1.

Multi-stream plate fin heat exchangers are employed in the first pre-cooling cold box E1, the second pre-cooling cold box E2, and the subcooling cold box E3. Two hot streams with two cold streams, one hot stream with a plurality of cold streams, and two hot streams with a plurality of cold streams, are integrated in the first pre-cooling cold box E1, the second pre-cooling cold box E2, and the subcooling cold box E3, respectively.

In the first pre-cooling cold box E1, the two hot streams are: one stream of feed gas and one stream of external dry gas; and the two cold streams are: one stream of gas phase of the absorption tower T1 after heat exchange and warming by the subcooling cold box E3, and one stream of gas phase of the tower top separator V2 of the demethanizer T2 after heat exchange and warming by the subcooling cold box E3. In the second pre-cooling cold box E2, the one hot stream is the feed gas; and the plurality of cold streams are: one stream of gas phase of the absorption tower T1 after heat exchange and warming by the subcooling cold box E3, one stream of the liquid phase at the bottom of the absorption tower T1 after throttling and cooling, two streams extracted from a side of the demethanizer T2, one stream of −12° C. to −16° C. additionally added propane refrigerant, and one stream of −24° C. to −32° C. additionally added propane refrigerant. In the subcooling cold box E3, the two hot streams are: one stream of mixed phase of feed gas and part of the liquid phase after heat exchange and warming by the first pre-cooling cold box E1, and one stream of external dry gas after heat exchange and warming by the first pre-cooling cold box E1; and the plurality of cold streams are: one stream of gas phase in the tower top separator V2 of the demethanizer T2, one stream of gas phase, and one stream of gas phase at the top of the demethanizer T2.

### Example 2

Example 2 is an application of Example 1, and specifically is a recovery method using the ethane recovery system suitable for a rich gas with high carbon dioxide content as described above. The specific operation steps are as follows. A feed gas is divided into two paths, the first path of feed gas is pre-cooled by the first pre-cooling cold box E1 and then mixed with a part of a liquid in the low temperature separator V1. The mixed gas enters the subcooling cold box E3 for subcooling, and then is throttled and cooled before entering the upper part of the absorption tower T1.

The second path of feed gas enters the low temperature separator V1 after being pre-cooled by the second pre-cooling cold box E2, and all the gas phase separated by the low temperature separator V1 enters the first turbo expander expansion end K1 for pressure and temperature reduction and then enters the middle of the absorption tower T1. A part of the liquid phase separated by the low temperature separator V1 enters the bottom of the absorption tower T1.

A part of the external dry gas is subjected to heat exchange and cooling in the first pre-cooling cold box E1 and the subcooling cold box E3, and after being throttled and cooled, enters the top of the absorption tower T1.

The liquid phase at the bottom of the absorption tower T1 is divided into two paths after being pressurized in the first liquid phase pump P1. The first path of the liquid phase after being throttled and cooled enters the upper part of the demethanizier T2. The second path of the liquid phase after being throttled and cooled enters the second pre-cooling cold box E2 for heat exchange and warming, and then enters the middle of the demethanizier T2.

The gas phase at the top of demethanizier T2 is subjected to heat exchange and warming in the subcooling cold box E3, and then enters the tower top separator V2 of the demethanizier T2. The gas phase separated by the tower top separator V2 of the demethanizier T2 sequentially passes through the subcooling cold box E3 and the first pre-cooling cold box E1 for heat exchange and warming, and then passes through the output compressor K3 for pressurization, and finally is outputted after being cooled by the air cooler A1. The liquid phase separated by the tower top separator V2 of the demethanizier T2 is pressurized by the second liquid phase pump P2 and then enters the top of the demethanizier T2.

The gas phase at the top of the absorption tower T1 is divided into two paths after heat exchange and warming in the subcooling cold box E3. The two paths of gas phase merge after entering the first pre-cooling cold box E1 and the second pre-cooling cold box E2 for heat exchange and warming, respectively. The merged gas phase after being pressurized by the second turbo expander pressurized end K2 merges with the gas phase at the top of the demethanizier T2, followed by entering the output compressor K3 for pressurization. A condensate product at the bottom of the demethanizier T2 enters the subsequent fractionation processing units such as a dethanizer for processing.

In a case that the above ethane recovery system suitable for a rich gas with high carbon dioxide content and the recovery method therefor are in use, the components and working conditions of the feed gas are as follows.

Treatment scale of feed gas: 1000×104 m³/d, pressure of feed gas: 5.4 MPa, temperature of feed gas: 36° C., and pressure of dry gas export: 5.4 MPa.

The composition of feed gas is shown in Table 1 below.

TABLE 1

| Composition | N₂ | CO₂ | C₁ | C₂ | C₃ | iC₄ | nC₄ | iC₅ | nC₅ | C₆ | C₇ | C₈₊ |
|---|---|---|---|---|---|---|---|---|---|---|---|---|
| mol % | 0.56 | 2.5 | 84.4 | 8.39 | 2.25 | 0.63 | 0.57 | 0.27 | 0.12 | 0.13 | 0.12 | 0.06 |

With the temperature of feed gas of 36° C. and output pressure of dry gas of 5.4 MPa, the feed gas is divided into two paths. The first path of feed gas accounts for 21.5% of the total feed gas volume. The first path of feed gas, after being pre-cooled to the temperature of −54.5° C. and the pressure of 5.36 MPa, is mixed into a part of the liquid in the low temperature separator V1. The liquid mixed accounts for 48% of the liquid phase volume at the bottom of the low temperature separator V1. The mixed gas enters the subcooling cold box E3 for subcooling, and then is throttled and cooled to the temperature of −92.6° C. and the pressure of 2.75 MPa, followed by entering the upper part of the absorption tower T1.

The second path of feed gas accounts for 81.5% of the total feed gas volume and is pre-cooled by the second pre-cooling cold box E2 to the temperature of −54.5° C. and the pressure of 5.36 MPa, followed by entering the low temperature separator V1. All the gas phase separated by the low temperature separator V1 enters the first turbo expander expansion end K1 for pressure and temperature reduction to the temperature of −76.7° C. and the pressure of 2.75 MPa, and then enters the middle of the absorption tower T1. A part of liquid phase is separated by the low temperature separator V1, which accounts for 52% of the liquid phase volume at the bottom of the low temperature separator V1, and enters the bottom of the absorption tower T1. A part of the external dry gas accounting for 10.5% of the total external dry gas volume is subjected to heat exchange and cooling by the first pre-cooling cold box E1 and the subcooling cold box E3, and then is throttled and cooled to the temperature of −98.8° C. and the pressure of 2.75 MPa, followed by entering the top of the absorption tower T1. The liquid phase at the bottom of the absorption tower T1 is divided into two paths after being pressurized by the first liquid phase pump P1. The first path of liquid phase accounts for 49% of the liquid phase volume at the bottom of the absorption tower T1, and the first path of liquid phase is throttled and cooled before entering the top of the demethanizier T2. The second path of liquid phase accounts for 51% of the liquid phase volume at the bottom of the absorption tower T1, and the second path of liquid phase is throttled and cooled, and then enters the second pre-cooling cold box E2 for heat exchange and warming to the temperature of −74.3° C. and the pressure of 3.25 MPa, followed by entering the middle of the demethanizier T2. The gas phase at the top of the demethanizier T2 is at the temperature of −87.5° C. and the pressure of 3.2 MPa, which passes through the subcooling cold box E3 for heat exchange and cooling to be at the temperature of −94.5° C. and the pressure of 3.18 MPa, followed by entering the top tower separator V2 at the top of the demethanizier T2. The gas phase separated by the top tower separator V2 at the top of the demethanizier T2 sequentially passes through the subcooling cold box E3 and the first pre-cooling cold box E1 for heat exchange and warming, and then passes through the output compressor K3 for pressurization to be at the pressure of 5.45 MPa and the temperature of 82.5° C., and finally is cooled by the air cooler A1 to be at the temperature of 40° C. and the pressure of 5.4 MPa and then is outputted. The liquid phase separated by the tower top separator V2 at the top of the demethanizier T2, after being pressurized by the second liquid phase pump P2, enters the top of the demethanizier T2. The gas phase at the top of the absorption tower T1 passes through the subcooling cold box E3 for heat exchange and warming to be at the temperature of −68.6° C. and the pressure of 2.68 MPa, and then is divided into two paths, which respectively flow into the first pre-cooling cold box E1 and the second pre-cooling cold box E2 for heat exchange and warming, and then merge. The merged gas phase is pressurized by the second turbo expander pressurized end K2 to be at the pressure of 3.13 MPa and the temperature of 39.6° C., and then merges with the gas phase at the top of the demethanizier T2, followed by entering the output compressor K3 for pressurization.

From the lower part of the demethanizer T2, two low-temperature liquid phases are extracted, one low-temperature liquid phase with a temperature of −56.8° C., and the other low-temperature liquid phase with a temperature of −70.5° C. The two low-temperature liquid phases, after being subjected to heat exchange and warming in the second pre-cooling cold box E2 and the subcooling cold box E3, respectively, flow into the demethanizer T2 and serve as a heat source for a side reboiler. At the bottom of the demethanizer T2, one liquid phase with a low temperature of −1.9° C. is extracted, which is subjected to heat exchange and warming in the second pre-cooling cold box E2 and then provides a heat source for a reboiler. A propane refrigeration system provides two temperatures of −37.28° C. and −14.12° C. for the second pre-cooling cold box E2, and the shaft power of the propane refrigeration system is 6312 kW.

The condensate product at the bottom of the demethanizier T2 enters subsequent fractionation processing units such as a dethanizer for processing, and the product is ethane, which has a recovery rate of 94%, a pressure of 3.2 MPa, and a temperature of 29.6° C.

The simulation results of this example show that: under the same working conditions, comparing with some of the existing dry gas recycle ethane recovery processes, the ethane recovery rate of the disclosure increases by 4.6%, the minimum freezing and blockage margin of CO₂ on the upper plate of the absorption tower T1 increases from 5° C. to 7° C., the amount of ethane products increases by 25.36 t/d, and the compression power of the system decreases by 236 kW. The compression power of the system is the sum of the compression power of the output compressor K3 and propane refrigeration compression power. The CO₂ freezing and blockage resistance capacity and ethane recovery rate of the ethane recovery device are improved, and the economic efficiency is significantly improved.

The above is based on the ideal example of the disclosure as a revelation, through the above description, the relevant staff can make various changes and modifications without departing from the scope of the technical ideas of the disclosure. The technical scope of the disclosure is not limited to the content in the specification, and must be determined according to the scope of claims.

1. An ethane recovery system suitable for a rich gas with high carbon dioxide content, comprising: a first pre-cooling cold box (E1), a second pre-cooling cold box (E2), a subcooling cold box (E3), a low temperature separator (V1), an absorption tower (T1), a tower top separator (V2) and a demethanizer (T2), wherein
a pre-cooling input end of the first pre-cooling cold box (E1) and a pre-cooling input end of the second pre-cooling cold box (E2) are in communication with an external feed gas, and a pre-cooling output end of the first pre-cooling cold box (E1) is in communication with a pre-cooling input end of the subcooling cold box (E3); a pre-cooling output end of the subcooling cold box (E3) is in communication with a top of the absorption tower (T1), and a pre-cooling output end of the second pre-cooling cold box (E2) is in communication with the low temperature separator (V1); and a gas phase end of the low temperature separator (V1) is in communication with a middle of the absorption tower (T1) via a first turbo expander expansion end (K1), and a liquid phase end of the low temperature separator (V1) is separately in communication with a bottom of the absorption tower (T1) and the pre-cooling output end of the first pre-cooling cold box (E1);
a gas phase end at the top of the absorption tower (T1) is in communication with a heat exchange input end of the subcooling cold box (E3), and a heat exchange output end of the subcooling cold box (E3) is separately in communication with a heat exchange input end of the first pre-cooling cold box (E1) and a heat exchange input end of the second pre-cooling cold box (E2); a heat exchange output end of the first pre-cooling cold box (E1) and a heat exchange output end of the second pre-cooling cold box (E2) are in communication with an input end of an output compressor (K3) via a second turbo expander pressurized end (K2), and an output end of the output compressor (K3) is in communication with an output end of an air cooler (A1); the output end of the air cooler (A1) is separately in communication with an outside and the pre-cooling input end of the first pre-cooling cold box (E1), and the pre-cooling output end of the first pre-cooling cold box (E1) is in communication with the pre-cooling input end of the second pre-cooling cold box (E2); the pre-cooling output end of the second pre-cooling cold box (E2) is in communication with the top of the absorption tower (T1), and the bottom of the absorption tower (T1) is in communication with an upper part of the demethanizer (T2) via a first liquid phase pump (P1); and a gas phase end at a top of the demethanizer (T2) is in communication with the heat exchange input end of the subcooling cold box (E3), and the heat exchange output end of the subcooling cold box (E3) is in communication with the tower top separator (V2); and
a gas phase end of the tower top separator (V2) is in communication with the heat exchange input end of the subcooling cold box (E3), and the heat exchange output end of the subcooling cold box (E3) is in communication with the heat exchange input end of the first pre-cooling cold box (E1); the heat exchange output end of the first pre-cooling cold box (E1) is in communication with the input end of the output compressor (K3), and a liquid phase end of the tower top separator (V2) is in communication with the upper part of the demethanizer (T2) via a second liquid phase pump (P2); and a condensate product discharge end is arranged at a bottom of the demethanizer (T2).

2. The ethane recovery system suitable for a rich gas with high carbon dioxide content according to claim 1, wherein the first liquid phase pump (P1) is in communication with the second pre-cooling cold box (E2) via an external pipeline and is in communication with the bottom of the demethanizer (T2).

3. The ethane recovery system suitable for a rich gas with high carbon dioxide content according to claim 1, wherein the bottom of the demethanizer (T2) is in communication with the second pre-cooling cold box (E2) via an external pipeline and is in communication with the bottom of the demethanizer (T2).

4. A recovery method using the ethane recovery system suitable for a rich gas with high carbon dioxide content according to claim 1, comprising the following specific operation steps: dividing a feed gas into two paths, with a first path of feed gas passing through the first pre-cooling cold box (E1) for pre-cooling, then mixing with a part of a liquid in the low temperature separator (V1), and a mixed gas entering the subcooling cold box (E3) for subcooling before being throttled and cooled, followed by entering the upper part of the absorption tower (T1);
a second path of feed gas passing through the second pre-cooling cold box (E2) for pre-cooling before entering the low temperature separator (V1), a gas phase separated by the low temperature separator (V1) entering the first turbo expander expansion end (K1) for pressure and temperature reduction before entering the middle of the absorption tower (T1), and a part of a liquid phase separated by the low temperature separator (V1) entering the bottom of the absorption tower (T1);
a part of external dry gas before being throttled and cooled passing through the first pre-cooling cold box (E1) and the subcooling cold box (E3) for heat exchange and cooling, followed by entering the top of the absorption tower (T1);
dividing the liquid phase at the bottom of the absorption tower (T1) into two paths after being pressurized by the first liquid phase pump (P1), with a first path of the liquid phase being throttled and cooled before entering the upper part of the demethanizer (T2), and a second path of the liquid phase being throttled and cooled before entering the second pre-cooling cold box (E2) for heat exchange and warming, followed by entering the middle of the demethanizer (T2);
the gas phase at the top of the demethanizer (T2) passing through the subcooling cold box (E3) for heat exchange and warming, followed by entering the tower top separator (V2) of the demethanizer (T2), the gas phase separated by the tower top separator (V2) of the demethanizer (T2) sequentially passing through the subcooling cold box (E3) and the first pre-cooling cold box (E1) for heat exchange and warming, and then passing through the output compressor (K3) for pressurization, and finally passing through the air cooler (A1) for cooling and then being outputted; and the liquid phase separated by the tower top separator (V2) of the demethanizer (T2) entering the top of the demethanizer (T2) after pressurization by the second liquid phase pump (P2); and
dividing the gas phase at the top of the absorption tower (T1) after heat exchange and warming in the subcooling cold box (E3) into two paths, which separately enter the first pre-cooling cold box (E1) and the second pre-cooling cold box (E2) for heat exchange and warming, followed by merging, and a merged gas phase after pressurization in the second turbo expander pressurized end (K2) merging with the gas phase at the top of the demethanizer (T2), followed by entering the output compressor (K3) for pressurization; and a condensate product at the bottom of the demethanizer (T2) entering subsequent fractionation processing units comprising a dethanizer for processing.

5. A recovery method using the ethane recovery system suitable for a rich gas with high carbon dioxide content according to claim 2, comprising the following specific operation steps: dividing a feed gas into two paths, with a first path of feed gas passing through the first pre-cooling cold box (E1) for pre-cooling, then mixing with a part of a liquid in the low temperature separator (V1), and a mixed gas entering the subcooling cold box (E3) for subcooling before being throttled and cooled, followed by entering the upper part of the absorption tower (T1);
a second path of feed gas passing through the second pre-cooling cold box (E2) for pre-cooling before entering the low temperature separator (V1), a gas phase separated by the low temperature separator (V1) entering the first turbo expander expansion end (K1) for pressure and temperature reduction before entering the middle of the absorption tower (T1), and a part of a liquid phase separated by the low temperature separator (V1) entering the bottom of the absorption tower (T1);
a part of external dry gas before being throttled and cooled passing through the first pre-cooling cold box (E1) and the subcooling cold box (E3) for heat exchange and cooling, followed by entering the top of the absorption tower (T1);
dividing the liquid phase at the bottom of the absorption tower (T1) into two paths after being pressurized by the first liquid phase pump (P1), with a first path of the liquid phase being throttled and cooled before entering the upper part of the demethanizer (T2), and a second path of the liquid phase being throttled and cooled before entering the second pre-cooling cold box (E2) for heat exchange and warming, followed by entering the middle of the demethanizer (T2);
the gas phase at the top of the demethanizer (T2) passing through the subcooling cold box (E3) for heat exchange and warming, followed by entering the tower top separator (V2) of the demethanizer (T2), the gas phase separated by the tower top separator (V2) of the demethanizer (T2) sequentially passing through the subcooling cold box (E3) and the first pre-cooling cold box (E1) for heat exchange and warming, and then passing through the output compressor (K3) for pressurization, and finally passing through the air cooler (A1) for cooling and then being outputted; and the liquid phase separated by the tower top separator (V2) of the demethanizer (T2) entering the top of the demethanizer (T2) after pressurization by the second liquid phase pump (P2); and
dividing the gas phase at the top of the absorption tower (T1) after heat exchange and warming in the subcooling cold box (E3) into two paths, which separately enter the first pre-cooling cold box (E1) and the second pre-cooling cold box (E2) for heat exchange and warming, followed by merging, and a merged gas phase after pressurization in the second turbo expander pressurized end (K2) merging with the gas phase at the top of the demethanizer (T2), followed by entering the output compressor (K3) for pressurization; and a condensate product at the bottom of the demethanizer (T2) entering subsequent fractionation processing units comprising a dethanizer for processing.

6. A recovery method using the ethane recovery system suitable for a rich gas with high carbon dioxide content according to claim 3, comprising the following specific operation steps: dividing a feed gas into two paths, with a first path of feed gas passing through the first pre-cooling cold box (E1) for pre-cooling, then mixing with a part of a liquid in the low temperature separator (V1), and a mixed gas entering the subcooling cold box (E3) for subcooling before being throttled and cooled, followed by entering the upper part of the absorption tower (T1);
a second path of feed gas passing through the second pre-cooling cold box (E2) for pre-cooling before entering the low temperature separator (V1), a gas phase separated by the low temperature separator (V1) entering the first turbo expander expansion end (K1) for pressure and temperature reduction before entering the middle of the absorption tower (T1), and a part of a liquid phase separated by the low temperature separator (V1) entering the bottom of the absorption tower (T1);
a part of external dry gas before being throttled and cooled passing through the first pre-cooling cold box (E1) and the subcooling cold box (E3) for heat exchange and cooling, followed by entering the top of the absorption tower (T1);
dividing the liquid phase at the bottom of the absorption tower (T1) into two paths after being pressurized by the first liquid phase pump (P1), with a first path of the liquid phase being throttled and cooled before entering the upper part of the demethanizer (T2), and a second path of the liquid phase being throttled and cooled before entering the second pre-cooling cold box (E2) for heat exchange and warming, followed by entering the middle of the demethanizer (T2);
the gas phase at the top of the demethanizer (T2) passing through the subcooling cold box (E3) for heat exchange and warming, followed by entering the tower top separator (V2) of the demethanizer (T2), the gas phase separated by the tower top separator (V2) of the demethanizer (T2) sequentially passing through the subcooling cold box (E3) and the first pre-cooling cold box (E1) for heat exchange and warming, and then passing through the output compressor (K3) for pressurization, and finally passing through the air cooler (A1) for cooling and then being outputted; and the liquid phase separated by the tower top separator (V2) of the demethanizer (T2) entering the top of the demethanizer (T2) after pressurization by the second liquid phase pump (P2); and
dividing the gas phase at the top of the absorption tower (T1) after heat exchange and warming in the subcooling cold box (E3) into two paths, which separately enter the first pre-cooling cold box (E1) and the second pre-cooling cold box (E2) for heat exchange and warming, followed by merging, and a merged gas phase after pressurization in the second turbo expander pressurized end (K2) merging with the gas phase at the top of the demethanizer (T2), followed by entering the output compressor (K3) for pressurization; and a condensate product at the bottom of the demethanizer (T2) entering subsequent fractionation processing units comprising a dethanizer for processing.
